(19) United States
(12) Patent Application Publication
(10) Pub. No.: US 2022/0016160 A1
(43) Pub. Date: Jan. 20, 2022

(54) METHOD FOR PREVENTING OR TREATING INFECTION OF RESPIRATORY VIRUS UTILIZING GOLD NANOPARTICLES

(71) Applicant: Chur CHIN, Daegu (KR)

(72) Inventor: Chur CHIN, Daegu (KR)

(21) Appl. No.: 17/368,724

(22) Filed: Jul. 6, 2021

Related U.S. Application Data
(63) Continuation-in-part of application No. 16/929,730, filed on Jul. 15, 2020.

(51) Int. Cl. A61K 33/242; A61K 9/14; A61P 31/14

(52) U.S. Cl. CPC A61K 33/242; A61P 31/14; A61K 9/14

(57) ABSTRACT

A method of treating or preventing infection of a respiratory virus, includes administering to a subject a composition comprising gold nanoparticles, where the gold nanoparticles serve to hinder morphological change of protein of the virus and block entry of the virus into human cells. The gold nanoparticles in the composition are in form of negatively-charged gold colloidal solution. The composition further comprises an S1 proteolysis inhibitor. The gold nanoparticles each have a diameter of 0.1 nm to 100 nm.

### CROSS REFERENCE

This is a Continuation-In-Part of application Ser. No. 16/929,730 filed 15 Jul. 2020, which is now pending.

### BACKGROUND

The present invention relates to a method for preventing or treating infection of a respiratory virus with a composition comprising gold nanoparticles as an active ingredient.

Coronavirus disease (COVID-19) is an infectious disease caused by a newly discovered coronavirus. Most people infected with the COVID-19 virus will experience mild to serious respiratory illness. Coronavirus infection mainly results in a respiratory infection that has spread worldwide since it occurred in Wuhan, China in December 2019.

Coronavirus includes a spike protein, and a spike glycoprotein of coronavirus-19 mediates viral entry and membrane fusion to host cells, and thus has become a main target for vaccines and therapeutic products. However, so far, it was confirmed that most monoclonal antibodies (mAbs) do not neutralize synonymous mutations.

The spike glycoprotein of the coronavirus has a multifunctional molecular structure mediating coronavirus entry into host cells. First, the spike glycoprotein coronaviruses are known to induce viral attachment by recognition of various host receptors. Such a spike protein is present in the form of two structurally distinct pre-fusion and post-fusion morphologies.

### SUMMARY OF THE INVENTION

The present invention is contrived to overcome the conventional disadvantages. Accordingly, an objective of the present invention is to provide a composition with gold nanoparticles that serve to hinder the morphological change of corona virus, thus blocking its entry into human cells.

In order to achieve these and other objectives, the present invention provides a pharmaceutical composition for treating or preventing a respiratory virus, comprises gold nanoparticles.

The present invention provides a method of treating or preventing a respiratory virus, comprising administering a composition containing gold nanoparticles to a subject.

In one embodiment of the present invention, the gold nanoparticles serve to hinder the morphological change of a coronavirus protein and block the entry of a coronavirus protein into a cell.

In another embodiment of the present invention, the gold nanoparticles are included in the form of a negatively-charged gold colloidal solution.

In still another embodiment of the present invention, the composition further includes a S1 proteolysis inhibitor.

In yet another embodiment of the present invention, the composition is a composition for a nasal spray.

In yet another embodiment of the present invention, the gold nanoparticle has a diameter of 0.1 to 100 nm.

A composition according to the present invention contains gold nanoparticles as an active ingredient, and in the present invention, an effect of treating and preventing coronavirus according to the treatment of the gold nanoparticles was confirmed. The gold nanoparticles hinder the morphological change of a coronavirus protein, so that the entry of the coronavirus protein into a cell can be blocked, resulting in the prevention or treatment of virus infection.

### BRIEF DESCRIPTION OF DRAWINGS

FIG. 1 is a view showing the morphology of respiratory virus.

FIG. 2 is a view showing before and after fusion structures due to entry of the coronavirus into a viral host cell.

FIG. 3 is a view showing gold nanoparticles applied to the respiratory virus according to the present invention.

FIG. 4 is a view showing that gold nanoparticles are added to disulfide bonding between spike proteins of the respiratory virus.

FIG. 5 is a view showing a process of treating a coronavirus disease using a composition of the present invention binding to the virus.

FIG. 6 is a view showing the morphology of an S1 proteolysis inhibitor-binding gold nanoparticle.

FIGS. 7-18 are views showing experimental results.

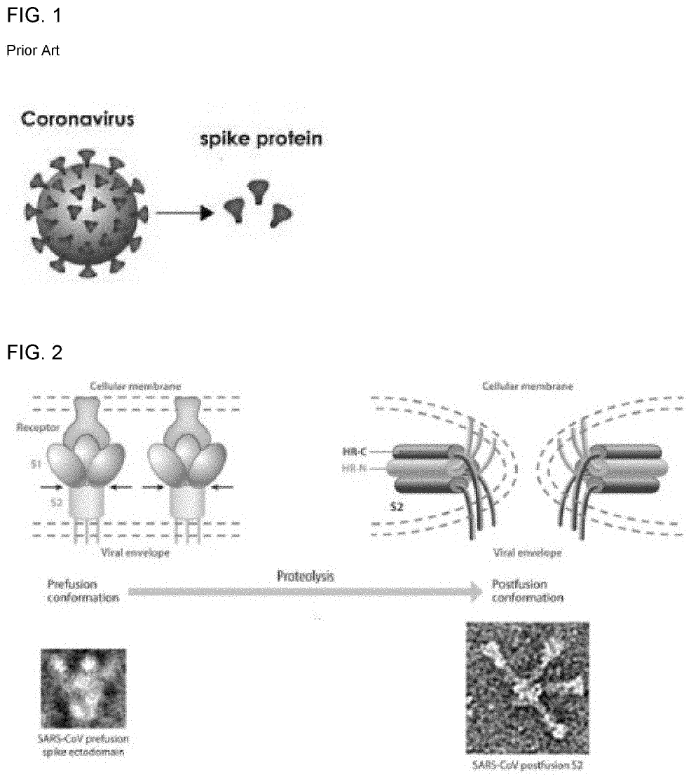

### DETAILED DESCRIPTION OF THE INVENTION

Referring to the accompanying drawings, the present invention will now be explained. A method of treating or preventing infection of a respiratory virus, comprises the step of administering to a subject a composition comprising gold nanoparticles, wherein the gold nanoparticles serve to hinder morphological change of protein of the virus and block entry of the virus into human cells.

Preferably, the gold nanoparticles in the composition are in form of negatively-charged gold colloidal solution. The composition may further comprise an S1 proteolysis inhibitor. The gold nanoparticles each have a diameter of 0.1 nm to 100 nm. The respiratory virus may be coronavirus (Covid-19). The respiratory virus may be SARS (Severe Acute Respiratory Syndrome) virus. The respiratory virus may be MERS (Middle East Respiratory Syndrome) virus. The respiratory virus is rhino virus. The respiratory virus is adenovirus. The respiratory virus may be influenza virus. The respiratory virus is echo virus. The respiratory virus is Coxsackie virus. The respiratory virus is respiratory syncytial virus.

The present inventor has recently developed nontoxic, broad-spectrum virucidal gold nanoparticles less than 10 nm in size, modified with sulfonic acids (mesilate) that mimic heparan sulfates. Camostat, a serine protease inhibitor can introduce gold nanoparticles to the influenza virus via ionic bonds. The inventor has examined the ability of a novel sulfonated colloid gold to inhibit the virus in vivo. Consequently, camostat-colloid gold is a promising candidate for the development of antiviral drugs to prevent and treat influenza infection.

Heparan sulfate proteoglycans (HSPGs) are highly sulfated and used by several viruses, for attachment to the cell surface. Because of the heavily sulfated chains, HSPGs present a global negative charge that can interact electrostatically with the basic residues of viral surface glycoproteins. HSPG-dependent ACE2 receptor viruses can be grouped as rhino virus, adenovirus, parainfluenza virus, echo virus, Coxsackie virus, coronavirus and respiratory syncytial virus (RSV). Gold nanoparticles coated with sulfonic acid inhibit different strains of influenza virus that do not bind HSPGs. The antiviral action is virucidal and irreversible for influenza A (H1N1, H3N2, and H5N1) and B virus strains. The present inventor designed antiviral nanoparticles with flexible linkers that mimicking HSPG. They allow effective viral association with a strong binding and multivalent units, generating forces that eventually lead to irreversible viral deformation.

Materials and Methods

Chemicals: Camostat mesylate powder was purchased from Hyperchem (Shanghai, China). Colloid gold (MediGOLD®) was purchased from Nutraneering (Irvine,Calif.,USA) and Tamiflu (oseltamivir phosphate) was purchased from Hoffmann-La Roche Limited (St. Louis, Mo., USA).

Mice and Viruses

All research studies involving the use of animals were reviewed and approved by the Institutional Animal Care and Use Committee (IACUC) at the Charles River Laboratory. Six to wight week old female C57BL/6 mice were housed (n=10) and the mouse-adapted influenza strainsA/PR/8/34 (H1N1; PR8) were provided by Charles River Laboratories. The viruses were amplified in 10 day old embryonated hen eggs using standard procedures.

Infection and Treatment of Mice

For primary influenza infection, the mice were anesthetized by intraperitoneal injection of Avertin (2,2,2-tribromoethanol; 240 mg/kg; Sigma-Aldrich, St. Louis, Mo., USA) before intranasal infection with 50 plaque-forming units (PFUs) of PR8 virus in 30 mL PBS (Wisent Incorporated). The mice were weighed daily and assessed for clinical signs of the disease. The mice were treated orally twice daily (b.i.d.) during seven days with saline or Tamiflu (oseltamivir phosphate; 5 or 50 mg/kg b.i.d.), camostat 30 mg/Kg), colloid gold (3 mL/Kg) to evaluate morbidity and adaptive immune responses or during 3 day for early cytokine, the immune response, and lung viral titers.

Organ and Cell Isolation

Bronchoalveolar lavages (BALs) were used to collect airway cells. Lung were mechanically processed and the cells were isolated through centrifugation and washing steps.

Lung Viral Titer Determination

ng viral titers were determined from 10-fold dilutions of clarified supernatants of lung homogenates in viral plaque assays. The number of viral plaques was counted to determine the viral PFUs.

Real-time PCR Analyses

RNA isolated from lung homogenates was reverse transcribed, and real-time PCR was performed. Fold inductions was calculated using the 2-DDC time method and normalized using ribosomal 18S RNA expression. Saline treated and uninfected mice served as the control.

Cell Surface Staining and Flow Cytometric Analysis

Fc receptors were blocked with anti-CD16/32 antibody (BD Biosciences, Franklin Lakes, N.J., USA) for 20 min at 4° C.

Monocytes/macrophages were identified. Lymphocytes were stained for 30 min at 4° C. After cell staining acquisitions were performed with a FACS Calibur or a FACS Can to flow cytometer, Flow cytometry data were analyzed using the Flow Jo 7.6.4 software (Tree Star, Ashland, Oreg., USA).

Cell Stimulation and Intracellular Cytokine Staining

Freshly isolated cells (13106/0.2 mL) were stimulated for 6 h with influenza NP366-374 or PA224-233 peptides (1 mM). The cells were subsequently stained for cell surface molecules, fixed, permeabilized, stained for intracellular IFNg, and analyzed using flow cytometry. Influenza specific CD8+ T cells were identified as IFN-g-producing cells.

Statistical Analyses

The results have been presented as the mean SEM. Data from the four groups were compared and analyzed using the unpaired Student's t test with Welch correction. For comparison of four groups, one-way ANOVA followed by Tukey's post test was performed. Statistical analyses were performed using Graph Pad Prism 5 (La Jolla, Calif., USA). A P value, 0.05 was considered statistically significant (*P<0.05, **P<0.01, and ***P<0.001) as shown in the drawings.

Result

Camostat-colloid gold administration reduces morbidity and decreases influenza replication in mice than Ostelmivir. Camostat was found to be effective in ameliorating mouse influenza by blocking hemagglutinin cleavage. In this study, mice were treated with camostat, camostat with colloid gold, oseltamivir and 50 PFU of PR8 virus. The mice that received camostat-colloid gold suffered no weight loss, whereas the mice treated Ostelmivir lost up to 20% of their initial body weight on day 6 post infection (d.p.i.). Lung consolidation was significantly reduced in Camostatcolloid gold group compared to ostelmivir-treated mice on day 6 d.p.i. There was no difference in the survival rate at d 6 p.i. between ostelmivir-treated and camostatgold-treated groups. The clinical scores increased over the course of the study in the ostelmivir-treated group,but mostly remained at the base line level in camostatcolloid-gold treated mice. The symptom score consisted of 1) abnormal gestures of the body, 2) pilo-erection, 3) abnormal respiration, 4) eye discharge and 5) abnormal activity.

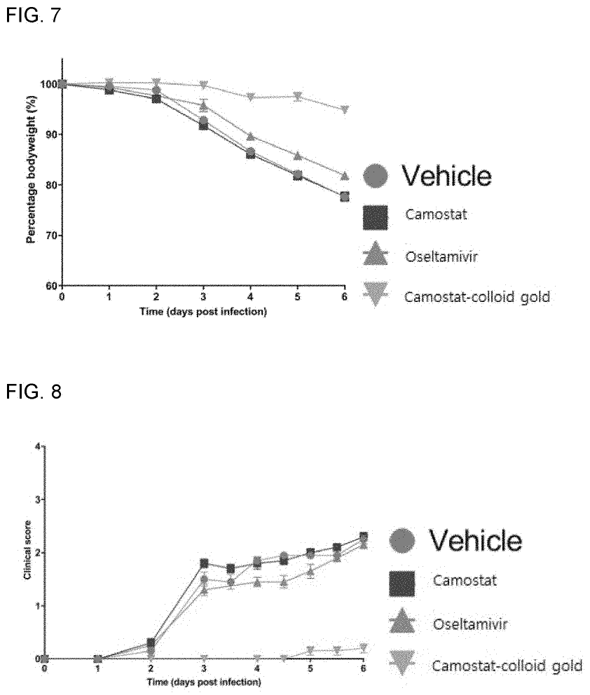

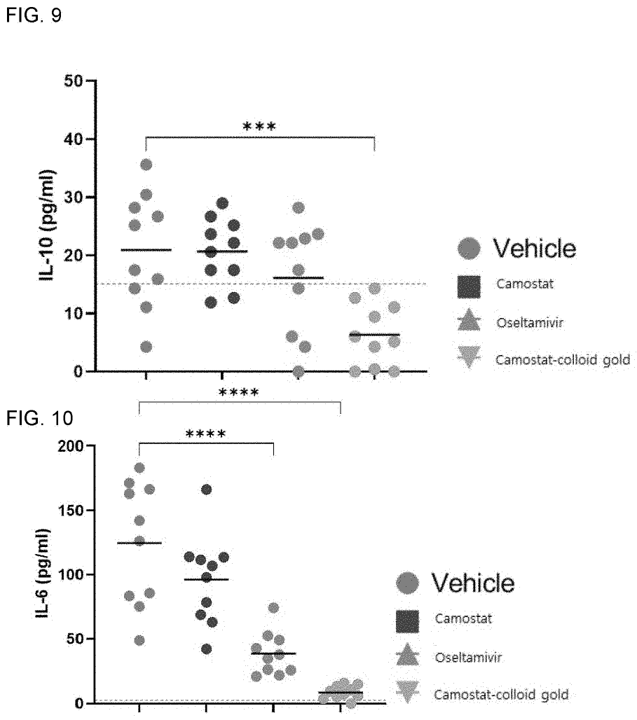

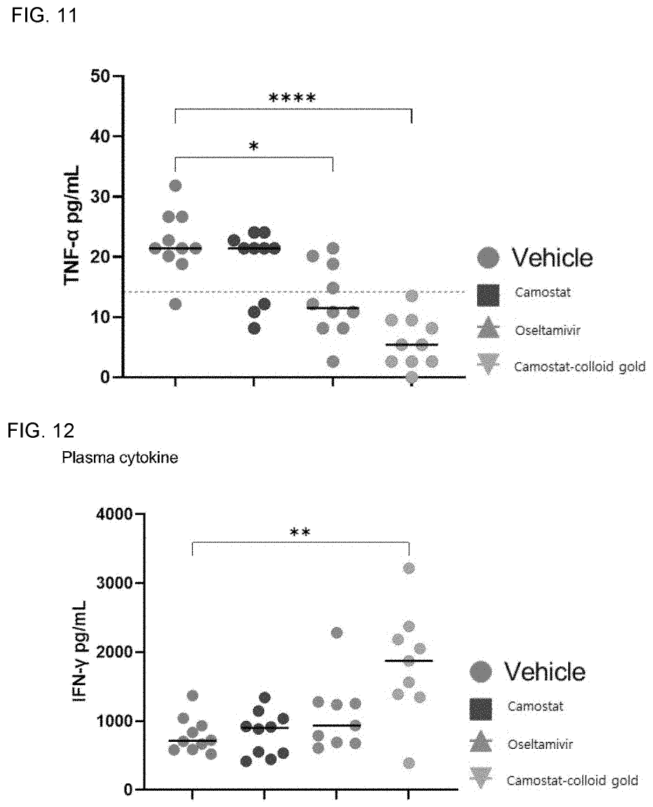

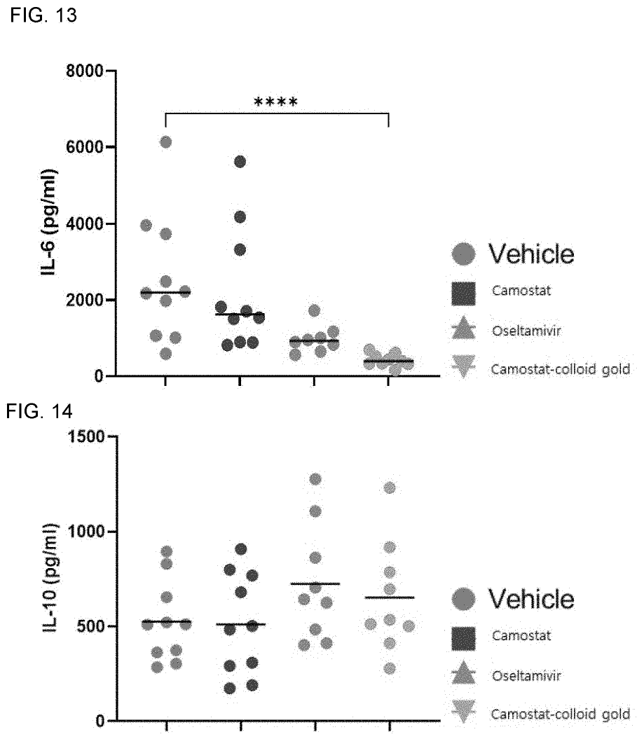

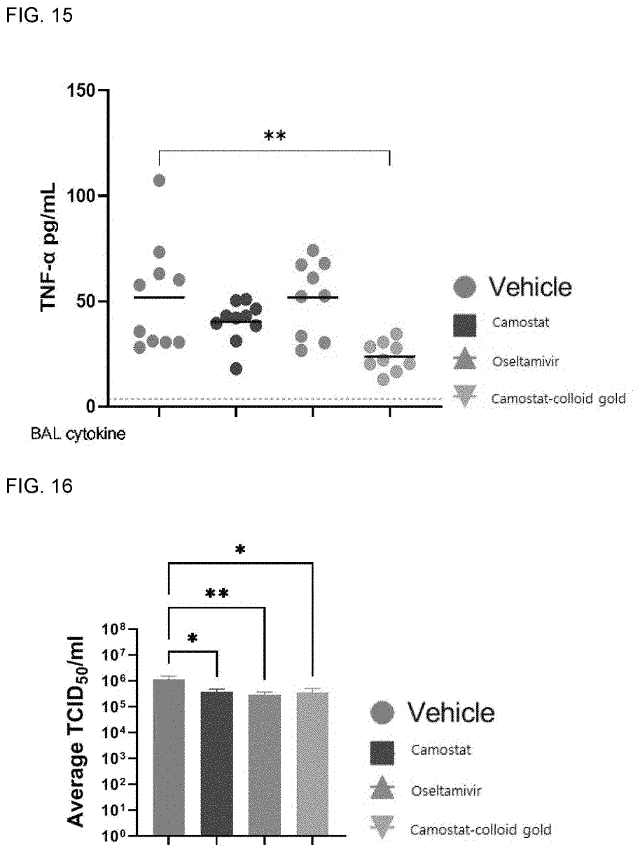

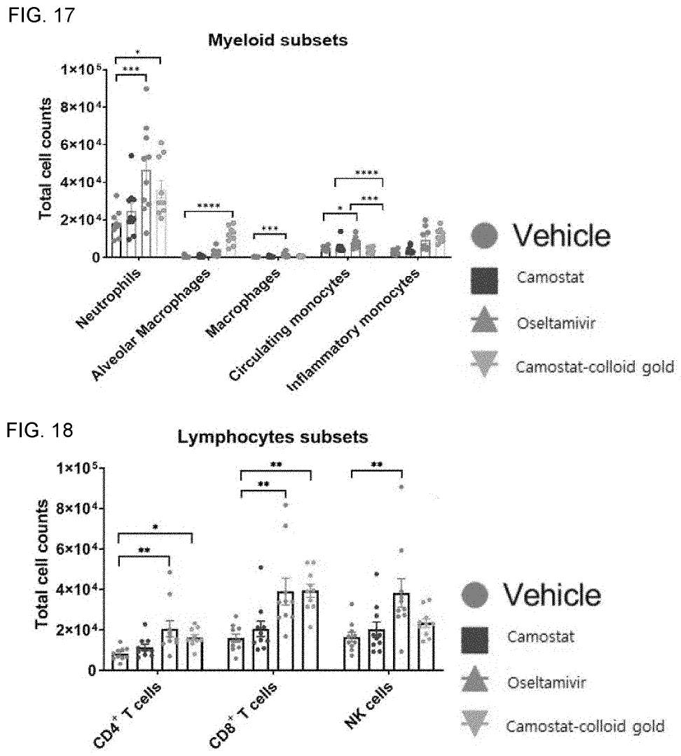

As shown in FIGS. 7-18, infection progressed with vehicle treated animals gradually losing body weight and showing increased clinical scores over the course of the study. camostat-colloid Gold reduced body weight loss, clinical score and lung consolidation in the animals. Camostat treatment did not significantly reduce disease progression compared to the vehicle. Oseltamivir treatment resulted in slightly improved clinical scores and survival.

Camostat-colloid gold administration reduced early inflammatory responses in mice during influenza infection compared to oseltamivir. In general, cytokines levels were higher in BAL than in plasma (5) (FIG. 2). Because of the significant effects of camostat-colloid gold administration on cytokine and chemokine expression, The present inventor subsequently determined its impact on the recruitment of leukocytes in the lungs as this process is highly dependent on inflammatory chemokines.

IL-6 (plasma and BAL), TNF-α (plasma and BAL) and IL-10 (plasma) were significantly decreased in the camostat-colloid gold group compared to the vehicle, indicating that the model worked. Oseltamivir induced a statistically significant decrease in IL-6 and TNF-α in plasma (p<0.0001 and p<0.05, respectively), although levels of TNF-α were predominantly close to or below the functional base line. A decreasing trend was seen in IL-6 levels in BAL for the camostat group compared to the vehicle group, although this did not reach statistical significance. No statistically significant decreases in cytokine levels were seen for the camostat group. PR8 infection induced a massive recruitment of monocytes/macrophages, neutrophils to the lungs and airways of mice in camostat colloid gold and oseltamivir-treated mice. The lymphocyte influx into the BALF of influenza infected mice increased.

Camostat-colloid gold treatment resulted in a higher count of alveolar macrophages indicating protection against lung injury. The increased infiltration of inflammatory monocytes, CD4 and CD8 cells and T cells in the lungs of camostat colloid gold treated animals by day 6 is indicative of the effect of oseltamivir on slowing disease progression. When compared to vehicle, oseltamivir increased cellular infiltration into the lung for all the cell subsets analyzed except for alveolar macrophages. An increasing trend was seen in the alveolar macrophage count in the BAL in the oseltamivir group compared to that in the vehicle group indicating possible protection against lung injury, although this did not reach statistical significance.

Camostat treatment did not alter immune cell infiltration in the lungs compared to that in the vehicle. What was found is that camostat, camostat-colloid gold and osteltamivir caused significantly reduction in lung viral titers.

Camostat-colloid gold significantly lowered viral load at day 6. Both camostat treatment and oseltamivir significantly reduced the viral load in the lungs at day 6 with camostat-colloid gold. Camostat helps ferry gold nanoparticles from the receptor binding sites to the glycoprotein of influenza virus. Gold nanoparticles which are ideal antivirals target conserved viral domains and are virucidal, These properties of gold nanoparticles make them as effective antiviral inhibitor.

The present invention provides that gold nanoparticles hinder a morphological change of a coronavirus protein, to block the entry of a coronavirus protein into a cell, resulting in inhibition of the cellular entry of the coronavirus.

The present invention also provides a pharmaceutical composition for treating or preventing infection of a respiratory virus, comprising gold nanoparticles.

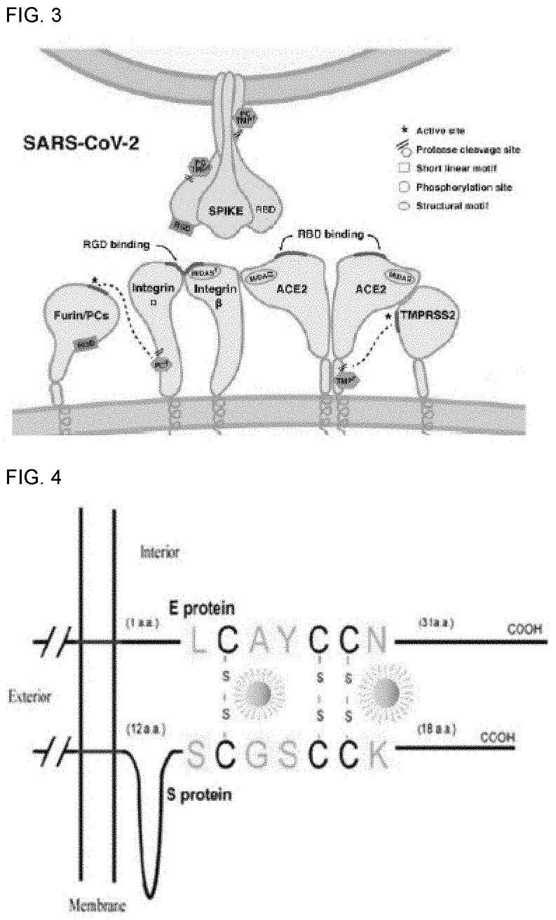

In FIG. 3, the gold nanoparticles serve to hinder the morphological change in a coronavirus protein to block the entry of the coronavirus protein into a cell. According to such blocking, for example, COVID-19 may be treated or prevented. In further detail, as shown in FIG. 4, a negatively-charged colloidal gold solution may be inserted between a spike protein and an envelope protein. The insertion of the gold nanoparticles makes the morphological change of the spike protein difficult.

In the present invention, the gold nanoparticles may be provided in the form of a negatively-charged gold colloidal solution. When the gold colloidal solution is used through spraying into the nasal cavity, the viral entry into cells may be more effectively prevented, but there are no limitations on the administration method.

In the present invention, the gold nanoparticles each have a diameter of 0.05 nm to 100 nm, preferably 0.1 nm to 50 nm, more preferably 0.1 nm to 35 nm, and most preferably 0.1 nm to 10 nm.

The “coronavirus-19”, which is a causative virus of a disease to be prevented or treated by the composition of the present invention, is a novel type of virus first identified in Wuhan, China in 2019, and the genomic and protein information associated with the coronavrius-19 may be freely collected from a research information database and used.

The term “prevention” used herein refers to all actions of suppressing COVID-19 or delaying the onset thereof by administration of the pharmaceutical composition according to the present invention.

The term “treatment” used herein refers to all actions involved in alleviating or beneficially changing symptoms of COVID-19 by administration of the pharmaceutical composition according to the present invention.

The present invention provides the simplest method of blocking the entry of a spike protein into a cell by hindering the morphological change of the protein using negatively-charged colloidal gold entering from an endotracheal tube or a nasal sprayer to the lungs. Here, small amounts of gold particles may migrate into the systemic circulatory system (10 nm to 40 nm).

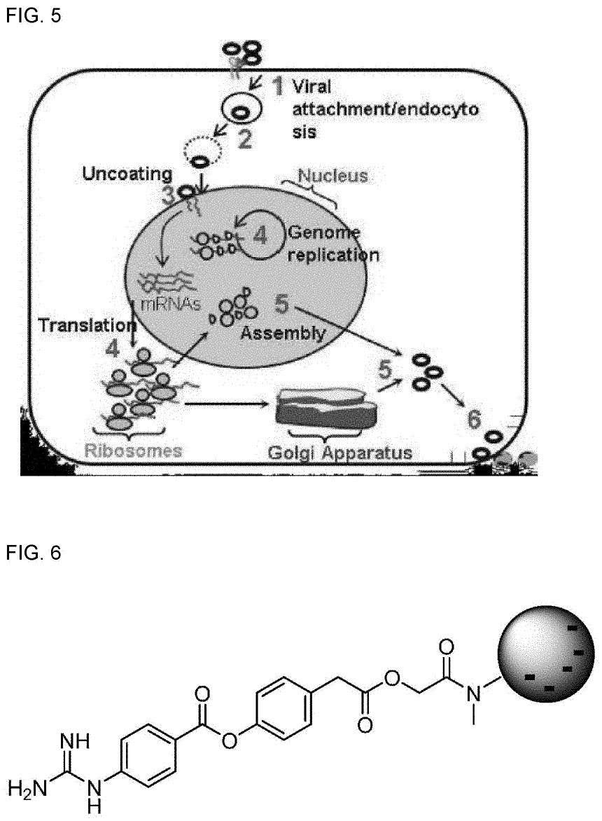

FIG. 5 shows that, when replicated coronavirus-19 is released from lung cells, a negatively-charged colloidal gold nanoparticles may easily bind to the positively-charged replicated virus, the virus may then be removed by phagocytosis, and thus the progression of infection may be prevented.

In addition, the gold nanoparticles of the present invention may be used in combination with an S1 proteolysis inhibitor. As shown in FIG. 6, camostat, which is the S1 proteolysis inhibitor, and the negatively-charged gold nanoparticles may be combined by ionic bonding, and therefore, the efficacy of the S1 proteolysis inhibitor may increase. That is, the progression of infection caused by coronavirus-19 may be inhibited by bonding between the gold nanoparticles acting as a protein form blocker and the S1 proteolysis inhibitor. The S1 proteolysis inhibitor may be, as an example, camostat mesylate, but the present invention is not limited thereto.

The gold colloidal solution provided in the present invention refers to a colloidal solution or suspension of submicrometer-sized gold nanoparticles, present in a fluid, generally, water.

The pharmaceutical composition according to the present invention may include a pharmaceutically acceptable carrier in addition to the active ingredient. Here, the pharmaceutically acceptable carrier is commonly used in preparation, and includes lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate and mineral oil, but the present invention is not limited thereto. In addition, the pharmaceutical composition according to the present invention may further include a lubricant, a wetting agent, a sweetening agent, a flavor, an emulsifier, a suspending agent or a preservative, in addition to the above-mentioned components.

The pharmaceutical composition of the present invention may be orally or parenterally (e.g., intravenously, subcutaneously, intraperitoneally or locally) administered by a desired method, and a dosage may vary according to a patient's condition and weight, the severity of a disease, a dosage form, an administration route and duration, but may be appropriately selected by those of ordinary skill in the art.

The pharmaceutical composition of the present invention may be administered at a pharmaceutically effective amount. The “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s).

In consideration of all of the above-mentioned parameters, it is important to achieve the maximum effect with the minimum dose without a side effect, and such a dose may be easily determined by one of ordinary skill in the art. Specifically, the effective amount of the pharmaceutical composition of the present invention may be dependent on a patient's age, sex, condition and body weight, an absorption rate of the active ingredient in the body, an inactivation rate, an excretion rate, a type of disease, or a drug used in combination, and may be generally administered at 10 to 1000 mg/kg, preferably 50-750 mg/kg, most preferably 100 to 500 mg/kg of body weight daily or every other day, or divided into one or three doses. However, the effective amount may vary depending on an administration route, the severity of obesity, sex, body weight or age, and therefore, the scope of the present invention is not limited by the dose in any way.

In addition, the present invention provides a method of treating or preventing coronavirus infection or COVID-19, which includes administering a composition including gold nanoparticles to a subject. The term “subject” used herein refers to a target in need of treatment, and more specifically, a mammal such as a human or a non-human primate, a mouse, a rat, a dog, a cat, a horse, or a cow.

Furthermore, the present invention may provide a use of a composition including gold nanoparticles to treat or prevent coronavirus infection or COVID-19.

Hereinafter, to help in understanding the present invention, an exemplary example will be suggested. However, the following example is merely provided to more easily understand the present invention, and not to limit the present invention.

### EXAMPLES

To confirm the effect of the composition of the present invention, a composition including 2-nm gold particles were prepared. The effect of the composition including the gold nanoparticle was studied by intratracheal administration to an adult female mouse. As a result, 1 hour after the single administration, the administered gold nanoparticles were found in lung macrophages.

From the above result, it can be confirmed that the inert gold nanoparticles administered into the trachea are involved in phagocytosis mediated by lung macrophages.

That is, a negatively-charged colloidal gold solution may approach coronavirus which has positively-charged porosity and hinders the morphological change of S1 and S2 domains by covalent binding to a cysteine residue of a spike protein, demonstrating that coronavirus-19 particles that are not able to be fused to lung cells through viral attachment to a host receptor are likely to be phagocytosed by lung macrophages.

It should be understood by those of ordinary skill in the art that the above description of the present invention is exemplary, and the exemplary embodiments disclosed herein can be easily modified into other specific forms without departing from the technical spirit or essential features of the present invention.

Therefore, the exemplary embodiments described above should be interpreted as illustrative and not limited in any aspect.

The composition of the present invention is a pharmaceutical composition for treating or preventing coronavirus disease-19 (COVID-19), including gold nanoparticles, and may block the entry of a coronavirus protein into a cell by hindering the morphological change of the spike protein of the coronavirus when the gold nanoparticles are administered. Therefore, the composition may be effectively used in the field of treating a novel infectious disease, such as COVID-19.

1. A method of treating infection of a respiratory virus, comprising:
administering to a subject a composition comprising gold nanoparticles and camostat, wherein the gold nanoparticles and the camostat are ionically bonded, wherein the gold nanoparticles serve to hinder morphological change of protein of the virus and block entry of the virus into human cells.

2. The method of claim 1, wherein the gold nanoparticles in the composition are in form of negatively-charged gold colloidal solution.

3. The method of claim 1, wherein the camostat serves as an S1 proteolysis inhibitor.

4. The method of claim 1, wherein the gold nanoparticles each have a diameter of 0.1 nm to 100 nm.

5. The method of claim 1 wherein the respiratory virus is coronavirus (Covid-19).

6. The method of claim 1 wherein the respiratory virus is SARS (Severe Acute Respiratory Syndrome) virus.

7. The method of claim 1 wherein the respiratory virus is MERS (Middle East Respiratory Syndrome) virus.

8. The method of claim 1 wherein the respiratory virus is rhino virus.

9. The method of claim 1 wherein the respiratory virus is adenovirus.

10. The method of claim 1 wherein the respiratory virus is influenza virus.

11. The method of claim 1 wherein the respiratory virus is echo virus.

12. The method of claim 1 wherein the respiratory virus is Coxsackie virus.

13. The method of claim 1 wherein the respiratory virus is respiratory syncytial virus.

14. A method of treating or preventing infection of a respiratory virus, comprising:
administering to a subject a composition comprising gold nanoparticles and camostat mesylate, wherein the gold nanoparticles and the camostat mesylate are ionically bonded, wherein the gold nanoparticles serve to hinder morphological change of protein of the virus and block entry of the virus into human cells.
